(19) United States
(12) Patent Application Publication
(10) Pub. No.: US 2012/0039767 A1
(43) Pub. Date: Feb. 16, 2012

(54) SENSING ELEMENT FOR CYCLIC SATURATED HYDROCARBONS OPTICAL DETECTOR WHICH USES THE SAME

(75) Inventors: Kimio YOSHIMURA, Takasaki (JP); Teruyuki HAKODA, Takasaki (JP); Shunya YAMAMOTO, Takasaki (JP); Atsumi MIYASHITA, Takasaki (JP); Masaki SUGIMOTO, Takasaki (JP); Masahito YOSHIKAWA, Takasaki (JP)

(73) Assignee: JAPAN ATOMIC ENERGY AGENCY, Ibaraki (JP)

(21) Appl. No.: 13/180,579

(22) Filed: Jul. 12, 2011

(30) Foreign Application Priority Data
Jul. 16, 2010 (JP) 2010-161642

(51) Int. Cl. G01N 33/52

(52) U.S. Cl. 422/400; 422/50

(57) ABSTRACT

A sensing element for cyclic saturated hydrocarbons, which can optically detect in a short time the volatilized organic hydride at comparatively low sensing element temperature, and a detector which uses the sensing element are disclosed. The sensing element for cyclic saturated hydrocarbons has a transparent substrate made of ceramics such as quartz; a tungsten trioxide thin film having columnar structure on the transparent substrate, and a platinum layer formed to the thickness of 15 nm or less on the surface of the thin film by deposition.

### BACKGROUND OF INVENTION

The present invention relates to a sensing element for cyclic saturated hydrocarbons in which light transmittance changes when exposed to the atmosphere which contains cyclic saturated hydrocarbons, and an optical detector which detects the cyclic saturated hydrocarbons by using the sensing element.

The sensing element and the detector according to the present invention are able not only to be used in a hydrogen transport equipment and a hydrogen storage device which handles a large amount of cyclic saturated hydrocarbons, but also used to detect the leakage of these hydrocarbons from a stationary dehydrogenation equipment which supplies hydrogen to a domestic fuel battery and the leakage of these hydrocarbons from an on-board dehydrogenation equipment for a fuel cell vehicle. In addition, the present invention can be used as a leakage detector which secures the safety of the dehydrogenation equipment in all of machines which use hydrogen such as ships, trams, and boilers.

To realize the hydrogen society mentioned above, organic hydrides (cyclic saturated hydrocarbons such as cyclohexane and Decalin) by which the reversal occlusion and emission of hydrogen is performed through platinum catalyst have been researching and developing as a means by which the storage and the transport of hydrogen or fuel are safely carried out

Most of the conventional detectors which regularly detects flammable gases are of a contact-burning type or a heat conduction type as disclosed in JP 2005-207879 A. Because the power circuit which becomes an ignition source is necessary in these detectors, an explosion-proof type structure is required to evade the danger of ignition or explosion in the detector installation place. Therefore, the structure of the detector becomes a complex heavy objects, and thus the detector itself is expensive.

Moreover, the sensing element for the hydrogen gas having a similar structure to the sensing element for cyclic saturated hydrocarbons according to the present invention is disclosed in JP 2007-121013 A. The optical sensing device disclosed herein comprises a transparent substrate made of ceramics, a tungsten trioxide thin film formed on the transparent substrate by using sputtering, and a catalyst metallic layer such as platinum formed to the thickness of 50 nm from 30 nm on a surface of the thin film by deposition.

In JP 2007-121013 A, a method of measuring the hydrogen gas concentration by measuring the transmitted intensity of light by a photo detector is disclosed. This method is carried out based on the principle that the molecular hydrogen gas is adsorbed by catalyst metal, thereby hydrogen atom is dissociated, the dissociated hydrogen atom is diffused to tungsten trioxide thin layer of a foundation layer, and the thin layer colors, and light transmittance of the thin layer changes as a result.

### SUMMARY OF INVENTION

Because cyclic saturated hydrocarbons expected as a storage and transport medium of hydrogen is volatile and flammable, the leakage of the organic hydride must be promptly detected. Thus, the development of the detection method which can ensure safety is indispensable.

An object of the present invention is to provide a sensing element for cyclic saturated hydrocarbons, which can optically detect in a short time the organic hydride volatilized at comparatively low element temperature, and a detector which uses the sensing element.

The sensing element for cyclic saturated hydrocarbons according to one aspect of the present invention comprises: a transparent substrate made of ceramics such as quartz; a tungsten trioxide thin film having columnar structure on the transparent substrate, and a platinum layer formed to the thickness of 15 nm or less on the surface of the thin film by deposition.

The detector for cyclic saturated hydrocarbons according to another aspect of the present invention comprises: a sensing element for cyclic saturated hydrocarbons comprising a transparent substrate made of ceramics such as quartz; a tungsten trioxide thin film having columnar structure on the transparent substrate, and a platinum layer formed to the thickness of 15 nm or less on the surface of the thin film by deposition; a hollow heater which heats the sensing element to the temperature between 150° C. and 200° C., the light transmitted through the sensing element passing through the inside of the hollow heater; a housing having a gas inlet and a gas outlet, which accommodates the hollow heater and the sensing element, a part of each of opposing sides of the housing being composed of light transmissive materials, a light source, and optical measurement equipment provided with a photo detector for receiving the light emitted from the light source, wherein the change in light transmittance caused by the reaction of tungsten trioxide with hydrogen dissociated by platinum is measured by the optical measurement equipment when a cyclic saturated hydrocarbon compound-containing gas which inflows through the gas inlet is brought into contact with the platinum layer of the sensing element and is exhausted through the gas outlet.

As more fully described below, the transmitted intensity of light can be increased because the thickness of the deposition layer of platinum is thin and below half of the conventional deposition layer, and thus the signal-to-noise ratio can be improved. As a result, when the gas which contains a low concentration of cyclic saturated hydrocarbons leaks, the gas leakage can be detect in a short time.

### BRIEF DESCRIPTION OF DRAWINGS

FIG. 1 is a schematic view showing an example of a sensing element according to the present invention.

FIG. 2 is a block diagram showing an example of a detector according to the present invention.

FIG. 3 is a schematic view illustrating the basic principle of the sensing element according to the present invention.

FIG. 4 is a scanning electron microscope image showing the columnar structure of tungsten trioxide in the sensing element according to the present invention.

FIG. 5 is a graph showing results of a performance test for each gas concentration in the detector according to the present invention.

FIG. 6 is a graph showing results of a performance test for each operating temperature in the detector according to the present invention.

### DESCRIPTION OF THE PREFERRED EMBODIMENTS

The present invention provides a sensing element for cyclic saturated hydrocarbons comprises a transparent substrate made of ceramics, a tungsten trioxide thin film formed on the transparent substrate, and platinum deposited on a surface of the thin film, and a detector using the same. In the present invention, the sensing element having a platinum layer formed on a surface of the tungsten trioxide thin film formed on the transparent substrate which penetrates light within the range of infrared rays from visible radiation is used under heated conditions.

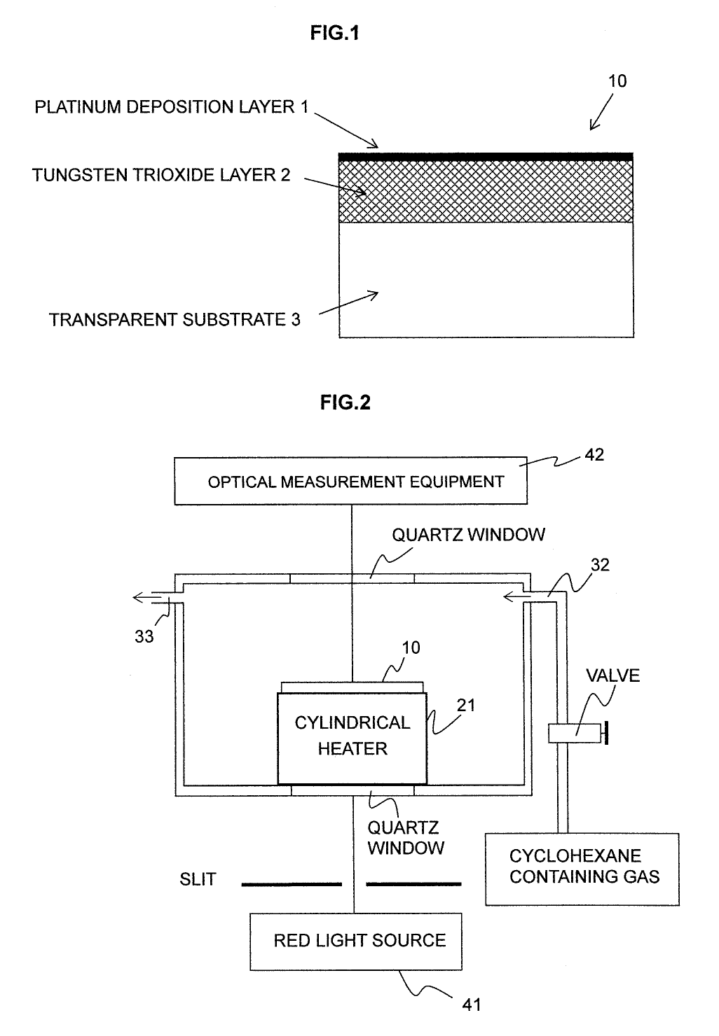

The structure of the sensing element according to the present invention is shown in FIG. 1. As understood from the sectional view shown in FIG. 1, the sensing element has platinum deposition layer 1, tungsten trioxide layer 2, and transparent substrate 3 in descending order. Cyclic saturated hydrocarbons are detected on the platinum deposition layer side. One example of a method of manufacturing the sensing element according to the present invention will be explained next.

A tungsten trioxide thin film was formed on a surface of the quartz substrate of 1 mm thick by using the radio frequency sputter. Using metallic tungsten as a target, a tungsten trioxide thin film was formed on the quartz substrate at substrate temperature 400-600° C. by sputtering the metallic tungsten target at 50 watts for one hour under the conditions of argon gas partial pressure 135 mPa and oxygen partial pressure 20 mPa. The thickness of the thin film formed was about 300 nm. Afterwards, platinum of 15 nm thick is formed on the tungsten trioxide thin film by using sputtering. The platinum sputtering was performed by using platinum metal as a target at electric power 50 watts for 40 seconds under the condition of argon gas pressure 135 mPa.

Cyclic saturated hydrocarbons to be measured by the sensing element according to the present invention is typically cyclobutane, cyclopentane, cyclohexane, methylcyclohexane and decalin, etc.

Next, the detector for cyclic saturated hydrocarbons according to the present invention will be explained referring to FIG. 2. In FIG. 2, reference numeral 10 designates a sensing element for cyclic saturated hydrocarbons explained in FIG. 1. Numeral 21 is a heater which heats sensing element 10 to a temperature between 150° C. and 200° C. Heater 21 is configured so that the light which penetrates the sensing element 10 can pass through the inside thereof. Reference numeral 31 designates a housing which accommodates heater 21 and sensing element 10. Housing 31 has gas inlet 32 and gas outlet 33, and a part of each of opposing sides of housing 31 is a window composed of light transmissive materials or quartz. Numeral 41 designates a red light source and 42 optical measurement equipment provided with a photo detector for receiving the red light given from red light source 41. Commercial items are available for these parts. Here, cyclohexane-containing nitrogen gas was used as a gas for detection for the test.

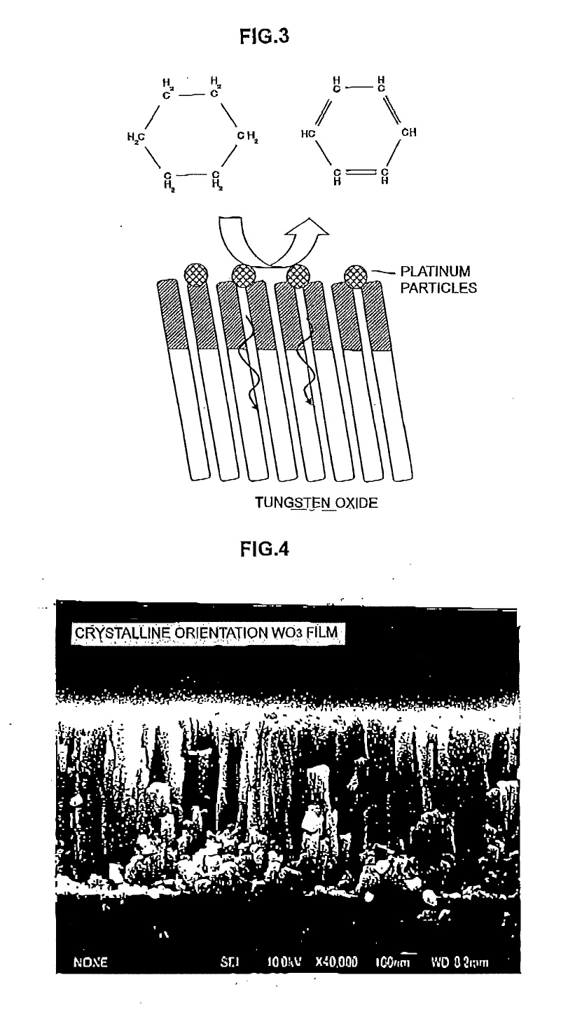

The change in light transmittance caused by the reaction of tungsten trioxide with hydrogen atom dissociated from the cyclic saturated hydrocarbons by platinum is measured by the optical measurement equipment when a cyclic saturated hydrocarbon compound-containing gas which inflows through gas inlet 32 is brought into contact with the platinum layer 1 (see FIG. 1) of the sensing element and is exhausted through gas outlet 33. The reacting state of hydrogen and tungsten trioxide is schematically shown in FIG. 3. This reacting state is not confirmed in experiment, but it would be expected to be the state shown in the figure by various studies. Moreover, to assist the understanding of the present invention, a scanning electron microscope image showing the crystalline orientation of a tungsten trioxide film (columnar structure) is shown in FIG. 4.

Cyclic saturated hydrocarbons can be detected by heating the substrate composed of tungsten trioxide and platinum according to the present invention. Although the substrate is heated by a ceramic heater, light sources such as infrared rays, pulsed lasers, etc. can be assumed to be a heat source. Tungsten trioxide-based sensing material can be manufactured easily only with making tungsten trioxide deposit on a surface of the transparent substrate.

The major ingredient of a tungsten trioxide thin film is tungsten trioxide. Preferably, the thickness of the thin film is 1 μm or less where flaking off is not caused from the transparent substrate. A tungsten trioxide thin film is formed by depositing metallic tungsten on a transparent substrate such as quartz under an oxygen atmosphere. Although the tungsten trioxide thin film is deposited by using a radio frequency sputtering method in the present invention, a direct current sputtering method, a laser ablation method, a vacuum evaporation method or a sol-gel method, etc. may be used. Moreover, although platinum is deposited on the surface of a tungsten trioxide thin film by using the radio frequency sputtering method, a direct current sputtering method, a laser ablation method, a vacuum evaporation method or a sol-gel method, etc. may be used.

Hereafter, the sensing element having tungsten trioxide of which the optical characteristic changes when exposed to the atmosphere which contains cyclic saturated hydrocarbons as a major ingredient, and the detection method of using the same will be explained in detail based on some tests.

Some characteristic tests of the sensing element were performed, and its performance was confirmed.

### Example 1

The change in optical characteristics of the gas which contains cyclic saturated hydrocarbons was evaluated at 200° C. by the measurement device shown in FIG. 2. The concentration 4.6% of cyclohexane diluted with nitrogen gas was used as cyclic saturated hydrocarbons for the evaluation. The red light of wavelength 645 nm was irradiated to the sample in a cell which can control atmospheres and temperatures, and the measurement was performed using spectrometer according to the following procedures.
(1) After the gas in a sample cell had been replaced with nitrogen gas for 5 minutes, it was heated to 200° C.
(2) Transmitted light intensity (Iₛ) of the sample was measured before exposed to cyclohexane.
(3) Concentration 4.6% of cyclohexane diluted with nitrogen gas was poured into the sample cell at a flow rate of 50 ml/min.
(4) Transmitted light intensity (I) of the sample was measured after exposed to cyclohexane gas.
(5) The change in light transmittance due to cyclohexane was evaluated by I/I₀.

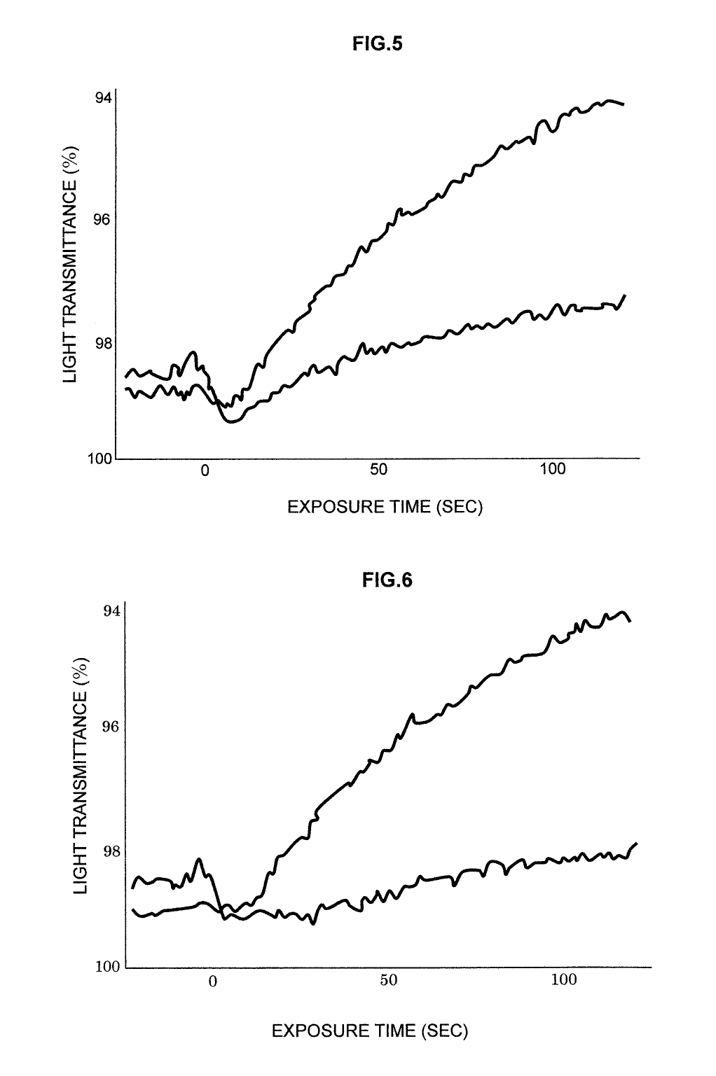

Time variation in light transmittance due to cyclohexane is shown in FIG. 5(a). The light transmittance has decreased according to an increase in exposure time to cyclohexane gas. That is, it is understood that coloring the tungsten trioxide layer has occurred. It is understood to be able to detect cyclic saturated hydrocarbons by observing the change in light transmittance as a result.

### Example 2

In the present invention, it is important to be able to detect cyclohexane even when the cyclohexane concentration is less than the lower bound (1.8%) of explosive limits in air. In connection with the sensing element, time variation in light transmittance due to concentration 1.1% of cyclohexane at 200° C. and at a flow rate of 50 ml/min was measured in a similar method to Example 1. The measurement results are shown in FIG. 5(b). It can be confirmed that the light transmittance has decreased according to an increase in exposure time to the cyclohexane gas. The observation was possible though the amount of change decreases compared with the case of concentration 4.6% of cyclohexane. It was understood to be able to detect even in the concentration 1.1% of cyclohexane as a result.

### Example 3

In the present invention, the heating temperature of the substrate is important. In connection with the sensing element, time variation in light transmittance caused due to concentration 4.6% of cyclohexane at 150° C. and at a flow rate of 50 ml/min was measured in a similar method to Example 1.

The measurement results are shown in FIG. 6(b). It can be confirmed that the light transmittance has decreased according to an increase in exposure time to the cyclohexane gas. However, the change was remarkably small compared with the case of substrate temperature 200° C. (see FIG. 6(a)). It was understood to be able to detect even in the concentration 1.1% of cyclohexane as a result. It was understood that the temperature of sensing element is important for the detection sensitivity as a result.

### Comparative Example 1

In the present invention, platinum on the tungsten trioxide thin film is important. The tungsten trioxide thin film layer where platinum is not deposited was made as a comparative example of the above-mentioned embodiment 1. The structure of the sensing element in comparative example 1 (not shown) is the same as the above-mentioned Example 1 except that platinum deposition layer 1 is not formed.

Time variation in light transmittance of comparative example 1 was also evaluated under the same conditions as Example 1. The light transmittance due to cyclohexane hardly changed in tungsten trioxide on which platinum is not deposited though the evaluation results are not shown in the figure. As a result, cyclohexane was not able to be detected. In a word, when the tungsten trioxide thin film is used to detect cyclohexane, a step of depositing platinum on the tungsten trioxide thin film becomes important.

As understood from the above-mentioned tests, it is very important to deposit platinum on tungsten trioxide thin film, and set the temperature of the substrate between 150° C. and 200° C., preferably to 200° C., in order to detect cyclic saturated hydrocarbons by using the tungsten trioxide thin film.

A detector which does not include a power circuit which becomes an ignition source or an explosion-proof component, etc. can be realized by using the present invention. Therefore, the present invention is useful for a portable cyclic saturated hydrocarbons detector, and a leakage detection system where optical fibers are used. The sensing element and the optical detector for cyclic saturated hydrocarbons, essential for practical use of the hydrogen energy for next-generation which can secure safety is provided according to the present invention.

What is claimed is:

1. A sensing element for cyclic saturated hydrocarbons comprises:
a transparent substrate made of ceramics;
a tungsten trioxide thin film having columnar structure on said transparent substrate, and
a platinum layer formed to the thickness of 15 nm or less on the surface of said tungsten trioxide thin film by deposition.

2. The sensing element for cyclic saturated hydrocarbons according to claim 1, wherein said transparent substrate is made of quartz.

3. The sensing element for cyclic saturated hydrocarbons according to claim 2, wherein said tungsten trioxide thin film is less than 1 μm in thickness.

4. A detector for cyclic saturated hydrocarbons comprises:
a sensing element for cyclic saturated hydrocarbons comprising a transparent substrate made of ceramics; a tungsten trioxide thin film having columnar structure on said transparent substrate, and a platinum layer formed to the thickness of 15 nm or less on the surface of said tungsten trioxide thin film by deposition;
a hollow heater which heats the sensing element to the temperature between 150° C. and 200° C., the light transmitted through said sensing element passing through the inside of said hollow heater;
a housing having a gas inlet and a gas outlet, which accommodates the hollow heater and the sensing element, a part of each of opposing sides of the housing being composed of light transmissive materials;
a light source, and
optical measurement equipment provided with a photo detector for receiving the light emitted from the light source,
wherein
the change in light transmittance caused by the reaction of tungsten trioxide with hydrogen dissociated by platinum is measured by said optical measurement equipment when a cyclic saturated hydrocarbon compound-containing gas which inflows through the gas inlet is brought into contact with said platinum layer of said sensing element and is exhausted through the gas outlet.

5. The detector for cyclic saturated hydrocarbons according to claim 4, wherein said tungsten trioxide thin film is less than 1 μm in thickness.
